(19) United States
(12) Patent Application Publication
(10) Pub. No.: US 2011/0309032 A1
(43) Pub. Date: Dec. 22, 2011

(54) SYSTEM AND METHOD FOR PHOTOCHEMICAL TREATMENT OF LIQUID SUBSTANCES WITH ULTRAVIOLET LIGHT INSIDE LIQUID CONVEYING TUBINGS

(76) Inventor: Markus MäkI, Pori (FI)

(21) Appl. No.: 13/203,123

(22) PCT Filed: Feb. 25, 2009
(86) PCT No.: PCT/FI2009/050153

§ 371 Date: Aug. 24, 2011

(51) Int. Cl. C02F 1/32; B01J 19/12; A61L 2/10; B01D 19/00

(52) U.S. Cl. 210/748.1; 95/46; 210/205; 250/435

(57) ABSTRACT

A system and method for photochemical treatment of liquid substances with ultraviolet light inside liquid conveying tubings. The system includes an elongated polymeric light guiding liquid conveyance tube having first and second open ends and an internal surface defining the interior and liquid conveying conduit of the tube, and a UV light source, which is optically connected to the interior and liquid conveying conduit of the tube. The light guiding tube includes one or multiple concentric layers of polymeric materials selected from a group that includes thermoplastic and thermosetting polymers, elastomers and composites. The tube has at least one UV light transmitting polymeric material layer having a lower refractive index value than the refractive index value of the liquid substance conveyed within the tube.

### FIELD

The present disclosure relates to the field of treatment of water and other liquid substances with ultraviolet (UV) light irradiation. More precisely, the aspects of the disclosed embodiments relate to method and system for UV processing of water and other liquid substances during conveyance within polymeric liquid conveying tubings.

### BACKGROUND

Ultraviolet (UV) light irradiation has been progressively employed for photochemical treatment of water, aqueous solutions, various other liquid substances and surfaces. In general, electromagnetic radiation in the UV range affects molecules, moieties and ions absorbing the radiation energy (photoexcitation) and is able to induce a diversity of photochemical reactions through various mechanisms within the molecular matrixes exposed to UV radiation. UV light irradiation is a highly versatile process presently employed for multiple purposes within many fields of industries. In the case of liquid processing, UV irradiation is commonly employed for molecular alteration and decomposition processes, the most typical applications including disinfection/sterilization and direct, indirect and photocatalytic photolysis of organic and inorganic target substances. Other applications of UV irradiation include for instance chemical synthesis, photocatalysis, UV curing of coatings and photo-polymerization.

In association with a diversity of equipment and devices within numerous fields of technologies and industries, wherein liquid substances critical to contamination, such as high purity water, aqueous solutions and other liquid substances holding potential for microbiological growth are conveyed through a diversity of different channels and tubings, problems in the microbiological quality and in the microbiologically derived chemical quality of the liquids are systematically confronted. In more detail, microbiological contamination of liquid substances and solid-liquid interfaces within liquid conduits appear in connection with numerous different processes and technical systems e.g. within the fields of ultra-pure water, medical, health-care and clean room technologies and pharmaceutical, semiconductor, chemical, food and beverages industries.

UV irradiation is one of the most powerful and feasible methods for liquid disinfection/sterilization process applications and it is in many cases highly preferred as a treatment method resulting from the fact that in association with irradiation treatment no chemicals are introduced to treated liquid substance and moreover, the process does not produce unfavorable by-products. UV irradiation and advanced combined UV disinfection processes are increasingly applied for inactivation of microorganisms (bacteria, viruses, molds, yeasts and protozoa) within liquid matrixes and UV irradiation for high purity surfaces.

The most common application for UV treatment of liquids is its employment for disinfection and sterilization of water as a primary method or typically as a part of multi step purification processes from small and medium scale high purity and ultra pure water (UPW) production to large scale industrial and municipal processes. In addition to disinfection and sterilization, other applications of UV irradiation within the field of water treatment include ozone reduction, chlorine and chloramine reduction, organic carbon (TOC, total organic carbon) reduction and reduction/inactivation of bacterial endotoxins. UV irradiation has been recently applied for multifunctional purposes as so called combination treatment methods combining UV irradiation with oxidants, such as ozone and hydrogen peroxide (advanced oxidation processes, AOPs) and photocatalysts (e.g. TiO₂) through which UV is used in addition to enhanced disinfection, e.g. for decomposition of refractory chemicals such as chlorofluorocarbons, metal complexes, taste and odor compounds, and other emerging contaminants. For various processes, preferred wavelengths and wavelength spectrums in the UV range produced with different types of light sources are utilized. The effect of UV irradiation on various molecular structures essentially depends on the wavelength and intensity of the employed radiation, and the exposure time of target material to UV (UV dosage).

One of the typical fluids, in which microbiological growth is a critical phenomenon, is ultra-pure water (UPW). Many industries suffer from the microbial contamination of UPW. These include the semiconductor, pharmaceutical, food & beverage, chemical and medical technology industries as well as healthcare facilities. Industrial UPW production is a complex multi-step process including typically a variety of steps (e.g., membrane filtration, UV disinfection and TOC reduction, vacuum degasification, heat treatment and ozonation) to inactivate microorganisms and to remove impurities. UPW manufacturing processes comprising various unit processes and their combinations enhance the ionic and organic chemical and microbiological water quality by decreasing e.g. total organic carbon (TOC) concentration, ion content, bacterial cell density and bacterial vitality, the result naturally depending on the process entity. It is however well established, that even UPW systems which produce water that meets ionic, microbiological and organic chemical standards are susceptible to microbial proliferation especially in association with circulation, storage and distribution. As in most high purity liquid systems, the primary source of bacterial contamination within UPW conduits are showed to be associated with bacterial biofilms (fouling) developing on solid surfaces in contact with UPW. The term biofilm in general is used of a layer covering a solid-liquid interface, which consists mainly of bacterial cells and extracellular polymeric substances excreted by the bacteria. One of the most vulnerable environments considering fouling derived microbial contamination in high purity fluid systems are untreated small to medium diameter tubes e.g. locating between various processes and delivering fluids to points of use.

In general, the central feature of UV irradiation of liquids is that the photochemical treatment process takes place only within the particular liquid volume through which UV light is propagated and at the solid surfaces exposed to UV light. Even though it is well established, that UV irradiation as a unit process is extremely efficient in disinfection and sterilization within the continuously irradiated UV reactor volume, its previously described momentary nature turns out to be a less favorable feature particularly associated with disinfection/sterilization applications. The untreated solid-liquid interfaces in the channels/tubings downstream the reactor chamber are again susceptible to microbial proliferation, originating from internal or external sources. During long operation periods, development of biofilm on the surfaces previously clean/sterile conduits can be expected, which leads to decrease in the microbiological and chemical quality of the treated liquid substances through release of microbial cells, parts of biofilm and bacterial components including endotoxins from the biofilm. A corresponding phenomenon is associated with other unit processes of liquid purification and disinfection, such as filtration processes (reverse osmosis, ultrafiltration, nanofiltration), pasteurization and distillation. In general, microbial control of the untreated conduits with chemical antimicrobial agents again is in most high purity applications limited or prohibited since they alter the chemical quality of the carrier liquid and may exhibit unfavorable properties. Combination of UV with oxidizing or photocatalytic species that can be increases the efficacy of the UV disinfection/sterilization, but it does not resolve the challenge of controlling the downstream conduits. The most common quality maintenance strategy for high purity liquid systems is based on periodical maintenance procedures, typically involving treatment of conduits, typically with hydrogen peroxide, steam or strong chemical disinfectant concentrations and installation of sterile replacement parts. However, disruption and removal of biofilms remains a challenge to UPW technology and presently, there is an increasing need for development of efficient continuous in-tube disinfection/sterilization and biofilm control technology for liquid transfer/distribution conduits.

As a unit process for treatment of liquid substances connected to various applications, typical conventional methods utilizing UV light for treatment of water/liquids have comprised employment of various types of UV reactors, wherein liquid detention time, reactor volume and wavelength spectrum and intensity of provided UV light are optimized according to the process carried out. The objective of the reactor design is to provide required UV light dose (unit mW/s/cm²) of the employed radiation for the process considered throughout the continuously replaced liquid volume inside the reactor chamber in an energy efficient way. Various UV lamp types with different reactor configurations are used. One of the most popular lamp types in UV disinfection applications are low-pressure mercury gas discharge lamps that peak mainly at 254 nm and 185 nm wavelengths. Recently application of various types of UV lasers and UV light emitting diodes (UV-LED) has more and more emerged to the field of disinfection.

Associated with the typical conventional UV reactor configurations, UV light sources, typically gas discharge UV lamp bulbs, are either submerged into treated liquid volume within reactor chamber, or alternatively positioned outside the reactor chamber and UV light is directed into the reactor chamber through windows, lenses or the like. To enhance UV reactor efficiency, various technical improvements have been introduced to increase UV dosage received by the treated liquids. These include e.g. increasing the number of lamp bulbs in a given volume, utilization of baffles and UV transparent coils. Recently, various configurations and types of reflecting surfaces and structures enabling total internal reflection (TIR) have been employed to the design of various reactor configurations in order to enhance the reactor efficiency. Reflection of UV light from the internal surfaces of the reactor chamber is preferred for maximizing the UV dosage given by a specific light source and to minimize the energy that is needed to provide required UV dosage.

In U.S. Pat. No. 6,773,584 a reactor configuration for UV treatment of water utilizing TIR and a flow tube is disclosed. The inlet and core of the cylindrical tank reactor unit is a transparent flow tube that is surrounded by a sealed, concentric volume of material having a lower refractive index than the fluid flowing in the flow tube, which enables TIR of UV light, when it is directed axially into the flow tube. International Patent Publication No. WO2005011753 discloses a method and reactor for in-line treatment of fluids and gases by light radiation comprising a tube or a vessel made of transparent material, preferably quartz glass, and surrounded by air, and having a fluid inlet, a fluid outlet, and at least one opening or window adapted for the transmission of light from an external light source into the tube. Air outside the tube or a vessel has a lower refractive index compared to the treated fluid, enables TIR.

Apart from the field of the present disclosure, technology, which is robustly related to the present disclosure has been employed recently in the field of optical chemical analysis, more precisely among long path absorption spectroscopy, wherein liquid core lightguides (LCL) are used as long path absorption cells. In this application the analyzed sample liquid is inserted as the core liquid into the LCL, required wavelengths are directed into the tube from the other end of the LCL and light intensity measured from the other end. Lightguide structures and chemical sensing techniques for this application are disclosed e.g. within U.S. Pat. Nos. 5,570,447 and 6,016,372. Typical structures of LCLs for light delivery applications are disclosed in association with several U.S. patents including Publications No. 5,546,493, 6,163,641, 6,507,688, 4,009,382 and 6,418,257. Most of previously mentioned patents include structures and light guiding strategies wherein low-RI materials have been employed.

### SUMMARY

The aspects of the disclosed embodiments provide a novel multi-applicable method and a system through which photochemical treatment of liquid substances with ultraviolet (UV) light is arranged during conveyance within elongated and more or less flexible polymeric light guiding liquid conveying tubings. Moreover, continuous direct UV irradiation of the internal surfaces of said liquid conveying tubings in accordance with the invention is provided. Furthermore, the aspects of the disclosed embodiments provide two additional processes combined to method and system derived from a structure alternative of the technology characteristic to the invention, namely degasification of liquid substances or dissolving of gas into liquid substances.

According to a first aspect of the disclosed embodiments, there is provided a method for photochemical treatment of a liquid substance with ultraviolet light, the method comprising using a polymeric tube for conveying said liquid substance, said tube having a first end and a second end, said tube comprising one or more concentric layers of polymeric materials such that said tube comprises at least one ultraviolet light transmitting polymeric material layer having a refractive index value, which is lower than the refractive index value of said liquid substance, passing said liquid substance into the interior of said tube through said first end by using said interior of said tube as a conduit for conveying said liquid substance between said first end and said second end, and directing said ultraviolet light through one or both ends of said tube into the interior of said tube so as to guide said ultraviolet light along said liquid substance by total internal reflection enabled by a difference between the refractive index of the liquid substance and the refractive index of said ultraviolet light transmitting material layer or layers, and photochemically treating said liquid substance in the tube by the ultraviolet light.

According to a second aspect of the disclosed embodiments, there is provided a system for photochemical treatment of a liquid substance with ultraviolet light, the system comprising a polymeric tube having a first end and a second end, said tube comprising one or more concentric layers of polymer materials such that said tube comprises at least one ultraviolet light transmitting material layer having a refractive index, which is lower than the refractive index of a liquid substance conveyed within said tube, said system further comprising a ultraviolet light source, which is optically connected to the interior of said tube so as to photochemically treat the liquid substance in the tube by passing ultraviolet light through one or both ends of the tube such that the ultraviolet light is guided along said liquid substance by total internal reflection enabled by a difference between the refractive index of the liquid substance and the refractive index of said ultraviolet light transmitting material layer or layers.

In accordance with the aspects of the disclosed embodiments, polymeric light guiding tubings are employed for liquid conveyance enabling in-tube treatment of conveyed liquid substances with UV light. In accordance with the method of the disclosed embodiments, an elongated polymeric tube is provided for conveying the liquid substance. The tube comprises one or multiple concentric layers of polymeric materials of which at least one layer comprises UV light transmitting (UV transmitting) polymeric material layer having a lower refractive index (RI) value than the RI value of the liquid substance conveyed within the tube. The polymeric materials are selected from a group comprising thermoplastic and thermosetting polymers, elastomers and composites. According to the method, the liquid substance is passed into said interior of said tube through first open end of said tube to be conveyed through the hollow tube interior and discharged through the second open end of the tube. UV treatment of conveyed liquid substance is carried out by providing UV light having a wavelength or wavelengths in a range from 100 nm to 400 nm and directing said UV light preferably axially through one or both open ends of the tube into the tube interior penetrating the conveyed liquid volume within said tube. UV light is guided along the tube through total internal reflection (TIR) enabled by RI value difference or differences between the conveyed liquid and one or more tube materials. UV light propagates through the light guiding liquid conveying tube over a significant length resulting in exposure of the liquid volume within the tube and the internal surfaces of the tube to UV radiation. The tube may comprise at least one outermost layer of UV light blocking material. Alternatively, the tube may comprise solely UV transmitting materials surrounded by preferably gas or alternatively liquid material having a lower RI than the conveyed liquid. In this case TIR is enabled by RI value differences between the liquid substance, tube material or materials and the gas or liquid material surrounding the tube having a lower RI value than the liquid substance conveyed within the tube.

In addition to UV treatment, concurrent processes of degasification or dissolving of selected gas into the liquid substance may be carried out during conveying of said liquid substance within the tube, by designing the tube to comprise one or more gas permeable materials of which at least one layer has a lower RI than the conveyed liquid, and by altering the composition and pressure of the gas volume surrounding the tube, resulting in gas transfer through the tube wall. Degasification is accomplished through creating vacuum atmosphere and dissolving of selected gas through creating an overpressure of gas selected to be dissolved within the tube surroundings.

The system corresponding to the method of the disclosed embodiments comprises an elongated polymeric light guiding liquid conveying tube and a UV light source, which is optically connected to the liquid conveyance conduit (tube interior) of said tube through one or both ends of the tube. Correspondingly, one or both tube ends or end portions of the tube are provided with means for accepting said liquid to be passed through the open tube end or ends and means for accepting UV light to pass through one or both open tube ends. In a preferred embodiment, the means are structurally provided in association with a connector structure included in the system, which is a solid, rigid structure of any suitable material connected and adapted to one or both ends of the light guiding liquid conveying tube, and which may be of its structural details and material composition, of various design.

In a preferred embodiment of the disclosed embodiments, UV light is produced in a light source distant to the light guiding liquid conveying tube, and UV light is delivered from the light source into the interior of said tube through a light cable. The connector structure is connected to one or both ends or end portions of the tube and in addition to previously described means, comprises means for attaching said tube and said distinct light cable to the connector structure, and at least one liquid conduit for providing a passage for said liquid substance between the tube interior and the exterior of the connector structure. Furthermore, the connector structure may further comprise means for correspondingly interfacing two or a plurality of said tubes and light cables delivering UV light into said tubes and multiple passages for one or more liquid substances. Accordingly, the connector structure may be incorporated to a construction of a device, an instrument or a technical system or the like.

In another preferred embodiment of the disclosed embodiments, UV light is produced in a light source, preferably in a UV-LED light source that is located in direct contact to the connector structure, or within the connector structure. Correspondingly, the connector structure comprises in addition to means for accepting said liquid to be passed into the tube interior for conveyance and means for accepting UV light to pass through one or both open tube ends, means for providing a support structure for the light source in contact to or within the connector structure, and at least one liquid conduit for providing a passage for said liquid substance between the tube interior and the exterior of the connector structure. Moreover, the connector structure may further comprise means for correspondingly interfacing two or a plurality of light guiding liquid conveying tubes and light sources and multiple passages for one or more liquid substances. Accordingly, the connector structure may be incorporated to a construction of a device, an instrument or a technical system or the like.

The aspects of the disclosed embodiments enable construction of elongated polymeric UV irradiation tubings/UV reactor tubings with optimized mechanical, thermal and optical properties. According to the system corresponding to the method of the disclosed embodiments, the light guiding liquid conveying tube comprises one or multiple concentric layers of more or less flexible polymeric materials of which at least one is a UV transmitting polymeric material layer having a lower RI value than the RI value of the liquid substance conveyed within said tube. Preferably, low refractive index fluoropolymers with high optical clarity and UV transmittance, such as Teflon AF (2,2-bis(trifluoromethyl)-4,5-difluoro-1,3-dioxole) exhibiting RI values down to 1.29, are employed. The polymeric materials are selected from a group comprising thermoplastic and thermosetting polymers, elastomers and composites. According to some embodiments of the present disclosure, the tube may comprise a single concentric layer of UV light transmitting polymeric material having a lower RI than the conveyed liquid, or two concentric layers of UV light transmitting polymeric materials of which the inner or outer layer comprises a material layer having a lower RI value than the opposite material layer. According to another embodiment of the present disclosure, the tube comprising one or multiple concentric UV transmitting polymeric materials further comprises at least one outermost layer of a UV light blocking material. According to one embodiment of the present disclosure, tube or a section of the length of the tube comprising solely UV transmitting layers, is positioned inside a solid UV light blocking outer structure forming a hollow volume between the exterior surface of said tube and said outer structure, said volume containing a transparent gas or liquid material having a lower RI value than the refractive index value of said liquid substance.

The system corresponding to the method with additional concurrent processes of degasification of the liquid substance or dissolving of selected gas into the liquid substance comprises a liquid conveying light guiding tube comprising concentric polymeric gas permeable material layers including at least one layer having a lower RI value compared to the RI value of the liquid substance. Preferably the tube comprises a single layer of gas permeable polymeric material having a lower refractive index value than the liquid conveyed within the tube, preferably Teflon® AF. The light guiding liquid conveying tube is enclosed within a solid outer structure leaving a sealed air or gas containing volume between the exterior of the tube and the solid outer structure. Degasification is carried out through providing a vacuum within the air volume surrounding the tube, resulting in simultaneous UV treatment and degasification of a liquid substance during its conveyance within the tube. Dissolving of gas into the liquid substance is carried out through creating overpressure of selected gas within the volume, resulting in simultaneous UV treatment of and dissolving of selected gas into a liquid substance during its conveyance within the tube.

The aspects of the disclosed embodiments enable arrangement of UV irradiation of liquid substances within polymeric liquid conveying tubings to induce various photochemical processes. Examples of photochemical processes include e.g. disinfection/sterilization of liquid substances, i.e. continuous microbial control of the conveyed liquid and tubing interior through primary UV irradiation or combined treatment methods (advanced oxidation, photocatalysts), other molecular alteration/breakdown processes including direct, indirect and photocatalytic photolysis of organic and inorganic substances and other photochemical processes such as photo induced chemical synthesis. Moreover, the present invention reveals the possibilities of incorporation of in-tube UV treatment to various devices and equipment within a variety of fields of industries and technologies. The aspects of the disclosed embodiments enables in certain limits, a continuous UV irradiation of the whole liquid volume of the conveyed liquid substance within the polymeric tube and the internal surfaces of the tube wall, which may result in a manifold, potentially a several log increase in UV dosage received by a particular volume of conveyed liquid through a particular amount of UV light energy, when compared to typical conventional UV reactors. Examples of potential applications for the disclosed embodiments include e.g. employment of the method and system for incorporation of more or less flexible polymeric UV reactor tubings to devices and equipment producing and supplying ultra pure and high purity water within e.g. manufacturing and packaging processes of pharmaceuticals, biotechnology products, chemical and biochemical reagents, clinical liquids, other standardized liquids, fine-electronics, semiconductor industry and in healthcare facilities, laboratories, clean rooms and medical equipment. In more detail, applications may include e.g. water transfer tubings between various unit processes incorporated to the high purity water production units and distribution and supply tubings of purified water between purification processes and the points of use. Moreover, potential applications include correspondingly incorporation of UV reactor tubings to various devices and equipment handling other liquid substances than water, such as chemicals and aqueous and chemical solutions and mixtures within e.g. fields of chemical, pharmaceutical and beverage industries, and medical, analytical and bioprocess technology. Thus, flexible, semi-rigid and rigid polymer tubes may be designed for the application of the invention. Associated with the application of the disclosed embodiments to UV disinfection, an elongated and more or less flexible polymeric conveyance tubings for liquid substances are provided, wherein sterilizing/disinfecting conditions predominate and biofilm formation onto the solid-liquid interfaces within the tubings is inhibited.

### BRIEF DESCRIPTION OF THE DRAWINGS

The following schematic drawings are illustrative and are not meant to limit the scope of the disclosed embodiments as encompassed by the claims. In the following, a general principle of the method and system, and some embodiments of the present disclosure will be described in simplified non-limiting illustrative examples. The appended drawings are part of the description. In the drawings,

FIG. 1 illustrates the general principle of the method and part of the corresponding system showing a cross sectional view of the UV light guiding liquid conveying tube, wherein the light guidance feature by total internal reflection of UV light is illustrated;

FIGS. 2-9 show cross sectional views of some examples of tube wall material structure alternatives for light guiding liquid conveying tubings according to the aspects of the disclosed embodiments, including examples of material layering order, TIR inferfaces and general light paths contributing to guidance of UV light;

FIG. 10 illustrates the general principle of the system, showing the general UV light path through the light guiding tube conveying a liquid substance;

FIG. 11 illustrates the general principle of one preferred embodiment of the system wherein UV light is produced in a light source distant to the UV light guiding liquid conveying tubing and delivered into the UV light guiding liquid conveying tube through a light cable, and wherein liquid substance, light cable and light guiding conveying tube are interfaced through a connector structure;

FIG. 12 illustrate the general principle of one preferred embodiment of the system wherein UV light is produced in a light source that is located in contact to a connector structure;

FIG. 13 illustrates of the general principle of one preferred embodiment of the system wherein UV light is produced in a light source that is located within a connector structure;

FIGS. 14-19 illustrate alternative general configurations of connector structures and their functions;

FIGS. 20-23 illustrate alternative general configurations of connector structures and outer structures;

FIG. 24 illustrates a cross sectional view of a gas permeable light guiding liquid conveying tube wall and a general principle of degasification of conveyed liquid;

FIG. 25 illustrates a cross sectional view of a gas permeable light guiding liquid conveying tube wall and a general principle of dissolving of gas into conveyed liquid;

FIGS. 26-28 illustrate alternative configurations of connector structures and outer structures and their functions associated with functions of degasification and dissolving of gas into conveyed liquid;

FIG. 29 illustrate a technical system comprising multiple separate devices, wherein the connector structure is incorporated into two of the devices and wherein UV light is produced in a light source distant to the UV light guiding liquid conveying tubing and delivered into the UV light guiding liquid conveying tube through a system of light cables; and

FIG. 30 illustrate a technical system comprising multiple separate devices, wherein the connector structure is incorporated into two of the devices and wherein UV light is produced in a light source that is located within a connector structure.

### DETAILED DESCRIPTION OF THE DISCLOSED EMBODIMENTS

The aspects of the disclosed embodiments provides a novel multi-applicable method and a corresponding system through which photochemical treatment of liquid substances with ultraviolet (UV) radiation is arranged during their conveyance within elongated polymeric light guiding liquid conveying tubings. The material structure of the tubing according to the method and the system is designed with an objective to guide UV light directed inside the tube interior along the tube, when it is filled with the conveyed liquid. The polymeric light guiding tubings are concurrently utilized in their traditional purpose; as transport channels for liquid substances, typically combined to various devices, equipment, instruments and technical systems. In association with the invention, photochemical treatment process of liquid substances with UV light is transformed from the traditional concept focusing on distinct tank reactor units into the interior of the flexible structure of polymeric tubings. Optimization of mechanical, thermal and optical properties of the tubing employed for particular application is enabled due to tube structure design and material selection.

In accordance with the aspects of disclosed embodiments, the term liquid substance is meant to cover practically all UV light transmitting liquid substances. Thus, the term is meant to cover any UV light transmitting pure compound, or a homogenous or a heterogenous mixture, such as a liquid, liquid colloid, liquid emulsion or liquid suspension, liquid solution or a mixture of any types of preceding liquid substances or the like. Typical examples include e.g. water (e.g. process water, rinse water, clinical water, dental unit water etc.), aqueous solutions and mixtures (e.g. various industry process liquids and liquid products, food and beverages industry liquids, medical liquids, analytical liquids, bioprocess liquids etc.) and liquid chemicals, chemical mixtures and solutions (e.g. various industry products, chemical reagents, solutions and mixtures).

In accordance with the aspects of disclosed embodiments, the term “photochemical treatment of liquid substances with UV light” is meant to cover any photochemical process induced through irradiation of said liquid substance with electromagnetic radiation in a range of from 100 nm to 400 nm to affect or alter any molecular property of said liquid substance. Examples of photochemical processes include e.g. photochemical UV disinfection/sterilization processes (primary UV irradiation and combined treatment methods such as advanced oxidation processes (O₃, H₃O, H₂O₂) and utilization of photocatalysts), and other molecular alteration and breakdown processes including direct or indirect photolysis of organic molecules (e.g. inactivation/breakdown of endotoxins, TOC reduction from water, dechlorination) or inorganic molecules (e.g. H₂O₂ and O₃ removal from water) and other photochemical processes such as photo induced chemical synthesis. More examples of potential applications for the invention are listed previously in association with the summary of the invention.

In accordance with the aspects of disclosed embodiments, now considering the employment of the aspects of disclosed embodiments for disinfection/sterilization and other potential applications utilizing antimicrobial wavelengths of UV light, a second beneficial process is simultaneously carried out in addition to UV irradiation of the conveyed liquid substances, namely continuous direct UV irradiation of the internal surfaces of the tubings (solid-liquid interfaces within the tube interior) resulting in inhibition of biofilm formation onto the surfaces.

In accordance with the aspects of disclosed embodiments, the light source employed for carrying out the photochemical treatment processes may be any suitable light source producing coherent or incoherent UV light. Light sources producing spot UV light are preferred. Light sources applicable for the invention include e.g. various laser UV light sources, gas discharge UV light sources (e.g. low and medium pressure mercury vapor gas-discharge lamps.) and UV light emitting diode (UV-LED) light sources. Lasers and LEDs are suggested as the most important light sources for current and future applications of UV irradiation technologies and presently, there exist many technical systems available on the market.

In accordance with the aspects of disclosed embodiments, polymeric light guiding tubings are employed for liquid conveyance enabling in-tube treatment of conveyed liquid substances with UV light. Referring to FIGA, showing a simplified schematic illustration of the general principle of the method according to the disclosed embodiments, an elongated polymeric light guiding tube 3 is provided for conveying a liquid substance 2, the tube having open first and second ends and an internal surface 9 defining the interior 8 of said tube. The liquid substance is passed into said interior of said tube 8 through first open end of said tube to be conveyed through the hollow tube interior 8 and discharged through the second open end of the tube. UV light 1 is directed through one or both open ends of the tube into the tube interior penetrating the conveyed liquid volume 2 that fills the tube interior 8 during conveyance. The light guiding feature of the tube is achieved by design of the tube material composition in accordance with the invention. According to the aspects of disclosed embodiments, the tube 3 comprises one or multiple concentric layers of more or less flexible polymeric materials of which at least one material layer has a lower refractive index (RI) value than the liquid substance conveyed within the tube. UV light 8 is guided through the tube through total internal reflection (TIR). TIR is enabled by RI value difference or differences between the conveyed liquid 2 and one or more UV transmitting tube materials, or by RI value differences between the conveyed liquid 2, one or more UV transmitting tube materials and gas or liquid material 7 surrounding the tube. Due to guidance of UV light along the direction of the axis of the tube, the volume of the conveyed liquid substance 2 and the internal surface 9 of the tube itself is objected to UV irradiation.

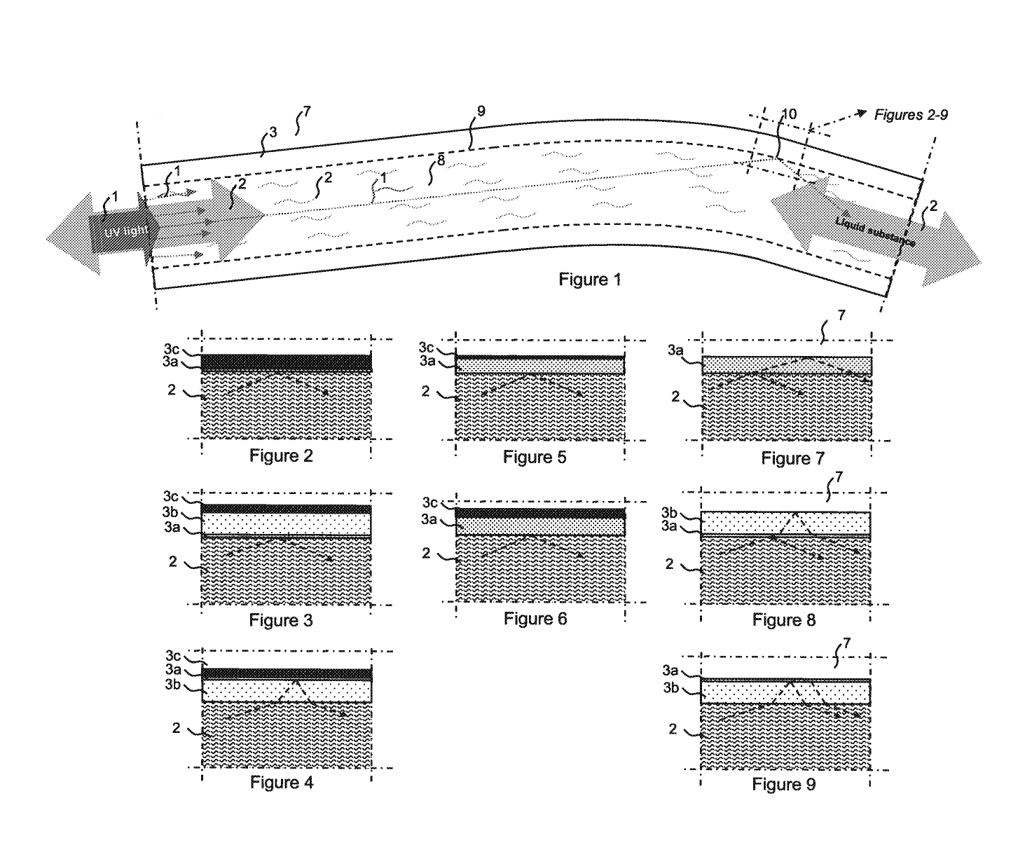

Referring to FIG. 1, the interface wherein TIR takes place depends on the structure, in more detail, concentric layer structure of the tube. Examples of tube structure alternatives according to the method and system, and corresponding TIR interfaces and light paths contributing to guidance of UV light are in more detail schematically illustrated in FIGS. 2-9, which are in more detail described in later chapters. To continue, it should be acknowledged, that the illustrated light path reflecting through TIR 10 illustrated as taking place at the internal surface of the tube 9 within said illustration in FIG. 1, refers only to certain tube material structure alternatives according to method and system, which turn out through illustrations in FIGS. 2-9.

In general, typical terminology used in association with general light guide technology include the terms “core” and “cladding”, which refer to the higher RI core and the lower RI cladding materials which enable guidance of light along the direction of the axis of a light cable through TIR. Thus, with terminology characteristic to light guide technology, the light guiding conveyance tube comprises at least one suitable cladding material within the tube body/wall, whereas the liquid that is conveyed through the interior of the tube, constitutes the light guide core.

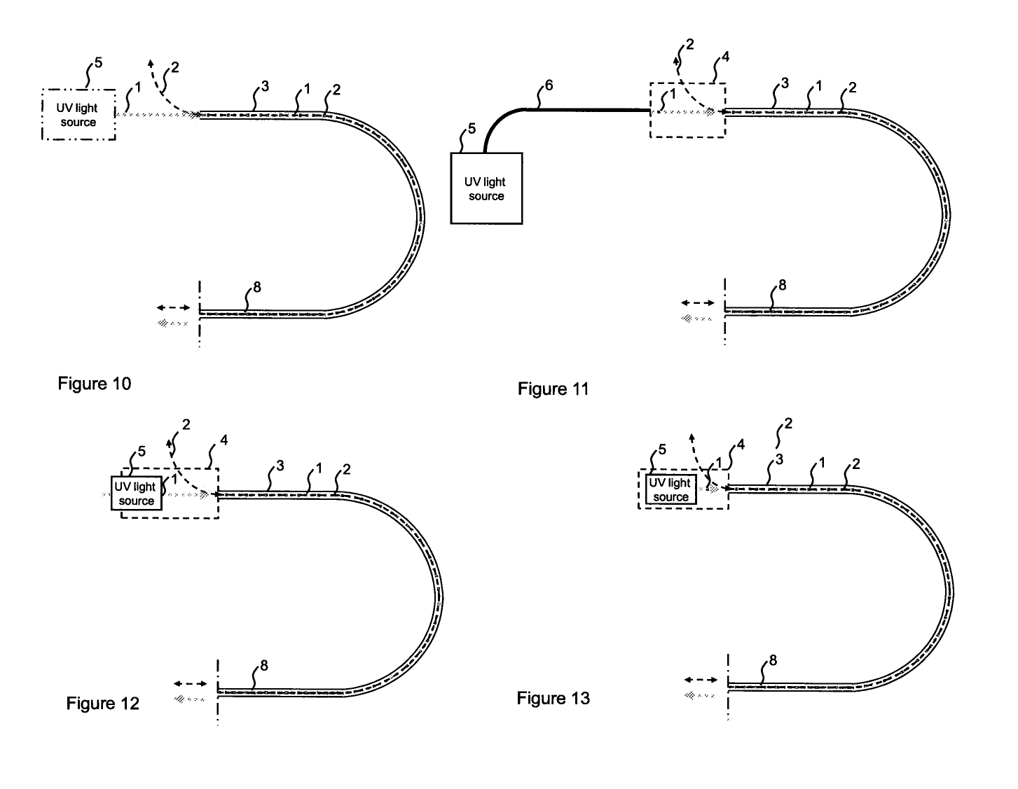

In accordance with the general embodiment of the present disclosure, now referring to FIG. 10, the system comprises an elongated polymeric UV light guiding liquid conveying tube 3 and a UV light source 5, which is optically connected to the interior (liquid conveying conduit) 8 of said tube 3 through one or both ends of the tube. Correspondingly, one or both tube ends or end portions of the tube are provided with means for accepting said liquid substance 2 to be passed through the open tube end or ends and means for accepting UV light 1 to pass through one or both open tube ends. The light guiding liquid conveying tube comprises one or multiple concentric layers of more or less flexible polymeric materials of which at least one layer comprises UV light transmitting (UV transmitting) material having a lower RI value than the RI value of the liquid substance conveyed within said tube.

The UV light guiding liquid conveying tubing, in accordance with the aspects of disclosed embodiments, employed for treatment of liquid substances with UV light, is hereon forth stated as UV irradiation tubing (from hereon forth stated in the text as UVI tube).

The specific tube wall structure of a UVI tube, may be constructed of several alternative light guiding strategies taking advantage of the principles of existing liquid core lightguide technology, and designed, according to the aspects of disclosed embodiments, to consist of one low RI material (single layer tube) or a combination of several materials (multilayer tube) that may comprise typically UV transmitting (UV transparent and UV translucent) low refractive index polymers, other UV transmitting polymers (such as a diversity of typical UV transmitting polymers having higher RI values than the RI value of water and UV transmitting polymers selected on the basis of their specific structural features, such as adhesive and antifouling properties) and opaque UV light blocking (non-UV transmitting) polymers. Furthermore, e.g. reflective material layers may be included in the structure to assist light guiding properties and adhesive layers to assist the material construction of the tube.

The structural and material design of a UVI tube according to the method and system of the disclosed embodiments is based on quantity and order of concentric polymeric material layers of varying structural and optical qualities. A UVI tube according to method and system may be either a single layer tube or a multilayer tube. The layer materials are selected from the group comprising thermoplastic polymers (thermoplastics), thermosetting polymers (thermosets), thermoplastic elastomers, thermosetting elastomers (elastomers), thermoplastic composites and thermosetting composites. Thus, the mechanical and thermal properties as well as processing properties of the UVI tube may be optimized due to various applications allowing design and employment of flexible, semi-rigid and rigid polymer tubes for the purpose of the invention. The mechanical and thermal properties of the UVI tube are primarily influenced by the number of the layers, the mechanical and thermal properties of employed polymers and diameters and thicknesses of particular layers.

Ultraviolet radiation may cause a photochemical effect within the polymer structure. Considering the invention in hand, the tube layers transparent to UV receive a continuous load of deep UV wavelengths. Hence the requirement of relatively high UV inertness is set for the materials utilized as the transparent layers to achieve long operation life. The same applies in some extent to the UV light blocking layers. Another preferred feature for material selection of the transparent inner layers includes high luminous transmittance (particularly UV light transmittance).

In a preferred embodiment, structures of UVI tubes according to the present disclosure involve using such material as one or more layers in the tube wall structures of UVI tubes, which has a refractive index (RI) lower than the refractive index of water (RI=1.333 at 589 nm and 25° C.). Some of the most preferable materials for this application belong commercial products of Teflon® AF (DuPont) family of amorphous fluoropolymers (Teflon® AF 2400; RI=1.29 at 589 nm and 25° C.). Teflon® AF exhibits a wide series of various favorable features referring to the invention in hand. These materials exhibit excellent optical clarity and light transmission features and smooth surfaces having the lowest RI-values of known polymers reaching down to 1.29 (Teflon® AF 2400). Furthermore, Teflon® AF polymers exhibit excellent physical, mechanical and chemical properties, including high strength, dimensional stability, gas permeability, creep resistance and thermal stability allowing end-use temperatures up to 300° C. In addition to other favorable features of Teflon® AF for the technology of the invention in hand, the said family of fluoropolymers also possesses outstanding gas permeability features. For Teflon® AF 2400, gas permeability values of 280,000 cB for CO₂, 99,000 cB for O₂, 220,000 cB for H₂ and 49,000 cB for N₂ have been announced. Furthermore, Teflon® AF polymers exhibit exceptional UV stability (do not deteriorate by deep-UV) and are chemically resistant to virtually all solvents and chemicals excluding few selected perfluorinated solvents used in processing of Teflon® polymers. To continue, Teflon® AF is reported to resist biofilm buildup and it can be used with the strongest cleaning solutions, chemicals and steam processes. In addition, these polymers are non-reactive and resist absorption of chemicals. Teflon® AF is highly recommended for use in pharmaceutical, biopharmaceutical and biotechnology processing equipment and high-purity fluid handling systems. Teflon® AF is also highly processable e.g. as fine coatings and thin films (e.g. spin, spray, brush, dipping) due to the limited solubility to perfluorocarbon solvents. Furthermore, Teflon® AF is processable with e.g. extrusion, pressing, injection molding into various pieces and objects/shapes, including various tubings. Another fluoropolymer having a slightly higher refractive index however being below the value of water is fluoroethylenepropylene (FEP). Other potential fluoropolymers with relatively low refractive indexes and variable mechanical and chemical as well as optical properties include e.g., perfluoroalkoxy (PFA), ethylenetetrafluoroethylene (EFTE), terpolymer of tetrafluoroethylene, hexafluoropropylene and vinylidene fluoride (THV), polyvinylidene fluoride (PVDF), polytetrafluoroethylene (PTFE). With varying mechanical and optical features, the unifying feature of all fluoropolymers is generally high chemical and UV resistivity.

The general design of the UVI tube structure according to the method and the system may be divided into two categories based to the existence of an outer UV blocking (non-UV light transmitting) material layer within the layer structure.

Within the first category, the UVI tube comprises one or more concentric inner polymeric layers of UV light transmitting polymeric materials which include at least one material layer having a lower RI value than the conveyed liquid, which are surrounded by one or more outer UV light blocking material layers. Thus UV light is insulated inside the UV light blocking outer layer or layers, within the inner optical zone of the tube enabling the guidance of light along the tube. According to one preferred embodiment of the invention, the tube comprises one or more inner layers of UV transmitting polymeric materials of which at least one layer comprises a material having a lower RI than the conveyed liquid substance and at least one outer layer of UV light blocking material. FIGS. 2-6 illustrate schematically some examples of material layer structures of UVI tubes belonging to this category. In the figures, layer 3a is a polymeric material layer having a lower RI value than the liquid substance 2, layer 3b is a UV transmitting polymeric material having a higher RI value than the liquid substance and layer 3c is a UV light blocking material. In practice, the UV light blocking material layer 3c may be a coating layer covering the outer surface of the tube (e.g. FIG. 5). Alternatively, the tube may be disposed inside an outermost UV light blocking sheath or outer tube which in practice forms the outermost UV light blocking material layer 3c. The sheath or tube may comprise any UV blocking material, which may be e.g. in a form of continuous solid material, fabric/textile or the like. (FIGS. 3, 4 and 6). Alternatively, the UV light blocking outermost layer 3c may comprise a tubular body of UV light blocking material of which internal surface is coated with one or more relatively thin layers of UV transmitting materials of which at least one has a lower RI than the conveyed liquid substance (FIG. 2). Through providing at least one UV light blocking outer layer, construction of tubings which insulate the surroundings of the tube body from UV irradiation is enabled. In one embodiment of the system comprising UVI tubes in this category, light guiding efficiency of the UVI tube may be assisted by incorporating UV light reflective material layers within the tube wall outside the UV transparent TIR enabling layer zone. Such assisting reflection can be enabled through e.g. materials forming reflective surfaces, such as UV enhanced aluminum, or thin UV transmitting polymer layers of which thickness is designed to correlate with the wavelength of light, enabling reflection through principles of thin film interference (phase change).

Within the second category, the UVI tube wall comprises solely of concentric layers of UV transmitting polymeric materials. According to a preferred embodiment, the UVI tube comprises one or multiple UV transmitting materials which include at least one material layer having a lower RI than the conveyed liquid. FIGS. 7-9 illustrate schematically some examples of material layer structures of UVI tubes belonging to this category. In the figures, layer 3a is a polymeric material layer having a lower RI value than the liquid substance 2, layer 3b is a UV transmitting polymeric material having a higher RI value than layer 3a and the liquid substance 2. In this case TIR within the tube is enabled by RI value difference between the conveyed liquid substance 2, UV transmitting tube material layer 3a having a lower RI than the conveyed liquid substance, and the material surrounding the tube, which may preferably be gas 7 or alternatively a liquid material having a lower RI than the conveyed liquid substance.

In an embodiment of the present disclosure, the UVI tube comprises in total two layers of materials which include an inner concentric layer of UV transmitting polymeric material having a lower RI value than the conveyed liquid followed by outer UV light blocking material layer. In a preferred embodiment, now referring to FIG. 2, the outer UV light blocking layer 3c is a concentric structural support layer of the tube, and the inner layer 3a consists of a thin coating of UV transmitting polymeric material having a lower RI value than the conveyed liquid substance 2. In a preferred embodiment, the inner layer 3a comprises a low RI fluoropolymer Teflon® AF or the like. Material of the outer layer may be selected from a diversity of suitable polymeric tube materials. In other words, the inner surface of a tube of selected material is coated with a low RI polymer, in a preferred embodiment with Teflon® AF or the like. In another preferred embodiment, now referring to FIGS, the outer UV blocking material layer 3c is a coating covering the external surface of a tube formed by the concentric inner structural support layer of UV transmitting polymeric material having a lower RI value than the conveyed liquid substance 2. In another preferred embodiment, now referring to FIG. 6, the outer UV light blocking material layer 3c is an outer tube or a sheath surrounding the inner tube that is formed by a concentric inner layer comprising UV trans-mitting polymeric material having a lower RI value than the conveyed liquid substance 2.

In another embodiment of the present disclosure, now referring to example illustrated in FIG. 3, the UVI tube comprises in total three concentric material layers which include one outermost layer of a UV light blocking material and two inner layers comprising concentric UV transmitting polymeric materials. The innermost layer 3a comprises a material having a lower RI value than the conveyed liquid substance and the second inner layer 3b comprises a material having a higher RI value than the innermost layer material 3a. In a preferred embodiment, the innermost layer 3a comprises a low RI fluoropolymer Teflon® AF or the like. In the case of the example, the second inner layer comprises a material having a higher RI value than the conveyed liquid substance 2 and it forms a structure support layer of the tube. The internal surface of the second inner layer is coated with a material having a lower RI value than the conveyed liquid substance 2, which forms the innermost layer 3a. The outermost UV blocking layer 3c may be a sheath, an outer tube or a coating surrounding the two UV transmitting material layers.

In another embodiment of the present disclosure, now referring to example illustrated in FIG. 4, the UVI tube comprises in total three concentric polymeric material layers which include one outermost layer of a UV blocking, UV light blocking material and two inner layers comprising UV transmitting materials. The second inner layer 3a comprises a material having a lower RI value than the conveyed liquid substance and the innermost layer 3b comprises a material having a higher RI value than the second inner layer material 3a. In a preferred embodiment, the second inner layer 3b comprises a low RI fluoropolymer Teflon® AF or the like. In the case of the example, the innermost layer 3b comprises a material having a higher RI value than the conveyed liquid substance 2 and it forms a structure support layer of the tube. The second inner layer 3a is a coating layer covering the external surface of the innermost layer 3b, the coating layer comprising a material having a lower RI value than the conveyed liquid substance 2. The outermost UV light blocking material layer 3c may be a sheath, an outer tube or a coating surrounding the two UV transmitting material layers. Material of the innermost layer 3b may be selected from a diversity of UV transmitting polymeric tube materials. Preferably a polymer having as high UV transmittance as possible is selected. Suitable materials include e.g. polyvinylidene fluoride (PVDF) and fluorinated ethylene propylene (FEP). Moreover, UV transmitting polymeric materials exhibiting antifouling properties to promote prevention of biofilm formation on the internal surface of the tube (e.g. antifouling polymer) may be employed.

In an embodiment of the present disclosure, now referring to FIG. 7, the tube wall structure of a UVI tube consists simply of a single layer 3a of UV transmitting polymeric material that has a lower RI value than the RI value of said liquid conveyed through the interior of said tube. In a preferred embodiment Teflon® AF or the like low RI material is used.

In another embodiment, the tube wall structure of the UVI tube consists of two concentric material layers; inner and outer layer. Now referring to FIG. 8, the outer layer 3b comprises a UV transmitting polymeric material having a higher RI than the inner layer 3a which comprises a UV transmitting polymeric material which has a lower RI value than the RI value of said liquid 2 conveyed within said tube. In association with probably the most feasible and economical configuration illustrated in FIG. 8, the outer layer 3b forms the body of the tube, and the inner layer comprises a thin coating. In other words, the inner surface of a tube of selected material is coated with a low RI polymer, in a preferred embodiment with Teflon® AF or the like low RI materials, depending on the application. In the case of the example illustrated in FIG. 8, the outer layer 3b has, in addition to having a higher RI than the inner layer material, a higher RI than the conveyed liquid. Material of the outer layer may be selected from a diversity of more or less UV transmitting, UV transparent or UV translucent polymeric tube materials, depending on whether TIR at the outermost surface of the tube is preferred. Suitable materials include e.g. polyvinylidene fluoride (PVDF), fluorinated ethylene propylene (FEP) polyethylene and silicone.

In another embodiment of the present disclosure, now referring to example illustrated in FIG. 9, the UVI tube wall structure of a UVIT consists of two concentric material layers; inner and outer layers. The outer layer 3a comprises a low RI polymer, having a lower RI value than the conveyed liquid, and the innermost layer 3b comprises a UV transmitting polymer having a higher RI than the outer layer material. In a preferred embodiment low RI fluoropolymer Teflon® AF or the like is used as the outer layer material. In association with probably the most feasible and economical configuration, as illustrated in FIG. 9, the inner material layer may form the tube body, and the outer layer may be a thin coating of low RI material, such as Teflon AF or the like. In the case of the example illustrated in FIG. 9, the innermost layer 3b has, in addition to having a higher RI than the inner layer material, a higher RI than the conveyed liquid. Material of the innermost layer 3b may be selected from a diversity of UV transmitting polymeric tube materials. Preferably a polymer having as high UV transmittance as possible is selected. Suitable materials include e.g. polyvinylidene fluoride (PVDF) and fluorinated ethylene propylene (FEP). Moreover, UV transmitting polymeric materials exhibiting antifouling properties to promote prevention of biofilm formation on the internal surface of the tube (e.g. antifouling polymer) may be employed.

In another embodiment of the present disclosure, the UVI tube comprises three or more concentric layers of UV transmitting materials, of which at least one layer consists of material having a lower RI value than the conveyed liquid, preferably Teflon® AF. The light guiding properties of the UVI tube may be optimized through layering order of specific materials. Additionally, UV transmitting antifouling materials may be employed as the innermost layer. The concentric material layers may be of various thicknesses from thin layers (e.g. coatings) to relatively thick layers able to simultaneously establish or assist in establishing the supporting structure of the tube. The objective of material selection in this case is to arrange the layers of selected materials in order to maximize the light guiding properties while taking into account the production cost assessment, and creating required mechanical properties of the tube with respect to application considered.

Characteristic to the general objective of the aspects of disclosed embodiments, previously described structure alternatives are intended to cover the general structure methodology of liquid core light guiding structures in a way, that alternative UVI tubing structures can be assessed when considering various technical and economical factors in connection with different application cases, manufacturing processes, costs, possible existing products applicable to utilized as UVI tubes, however, the main intention lying over that UVI tubes with different optical, functional and structural features and costs could be provided as tailored to various application areas.

In a preferred embodiment of the present disclosure, now referring to simplified schematic illustrations in FIGS. 11-19, the system includes a connector structure 4, which is a solid, rigid structure of any suitable material connected and adapted to one or both ends of the UVI tube, and which may be of its structural details and material composition, of various design. The connector structure provides structural means for accepting said liquid substance 2 to be passed through one or both open tube ends, and means for accepting UV light 1 to pass through one or both open tube ends to penetrate the tube interior and the volume of the conveyed liquid 2. In other words, the connector structure provides the structural configuration for channeling of the liquid between external site (source or destination) and the interior of the UVI tube, and the structural configuration through which UV light is delivered into the interior of the UVI tube. The structural and material design of the connector structure may naturally be diverse and thus the structure is characterized due to the primary functions that it provides. The connector structure may be a separate part tailored for various purposes, providing adaptations and connections with parts and liquid channels of larger process entities. Another purpose associated with the aspects of disclosed embodiments, is that functions of the connector structure may in practice be accomplished within the design of a diversity of various device configurations. Thus, the connector structure may be incorporated to a construction of a device, an instrument or a technical system or the like, such as liquid processing, purification or distribution equipment, dental units or packaging instrumentation, to name few. In this case the means provided by the connector structure are provided in association with and as part of a larger structure. Thus, it must be understood that, connector structure 4 (referring to FIGS. 11-19 wherein connector structure is illustrated with dashed line), illustrates a diversity of various constructions.

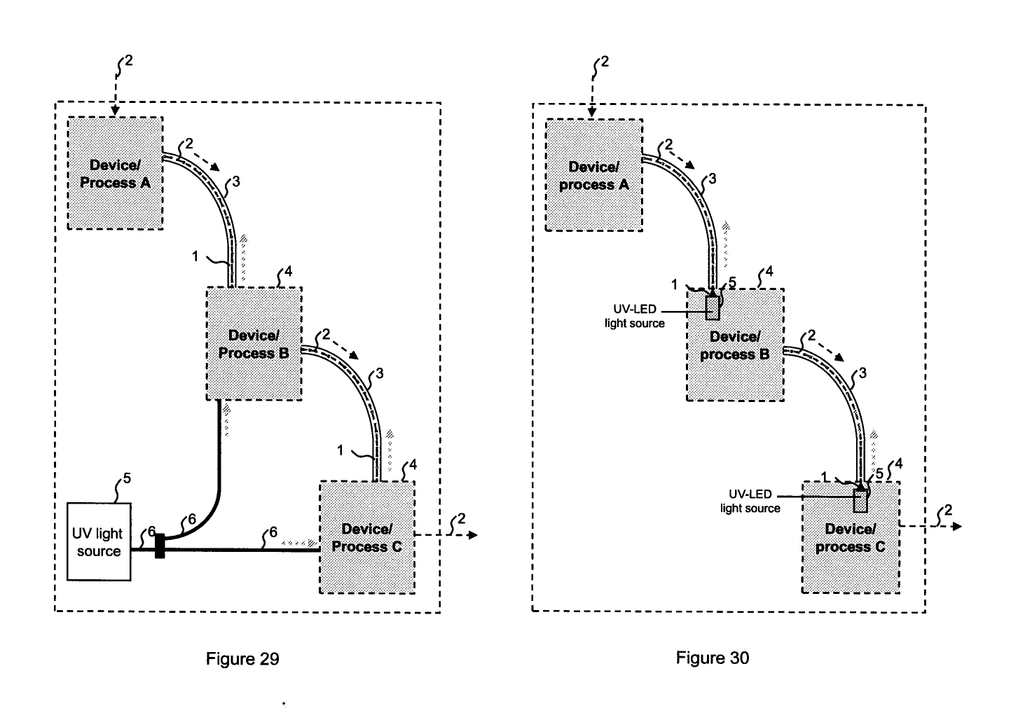

According to one preferred embodiment, now referring to FIG. 11, UV light is produced in a light source 5 distant to the UVI tube and delivered into the UVI tube through a light cable 6. Correspondingly, the connector structure 4 comprises in addition to means for accepting said liquid 2 to be passed into the tube interior 8 for conveyance and means for accepting UV light 1 to pass through one or both open tube ends, means for attaching said tube 2 and said distinct light cable 6 to the connector structure, and at least one liquid conduit for providing a passage for said liquid substance between the tube interior 8 and the exterior of the connector structure 4. In other words, the light-outlet end of the distinct UV light cable 6 that guides UV 1 from the light source 5 into the UVIT 3, is interfaced to the UVI tube 3 through a UV liquid connector structure 4. In the case of the embodiment, the means for accepting UV light 1 to pass into the interior 8 of the tube 3 that are provided in association of the connector structure 4, may for example comprise providing an optical window incorporated to the connector structure, or providing means for positioning the output end of the light cable at the open tube end in a way that the optical connection is achieved. Through this embodiment conduction of heat (that is associated with most UV light production technologies) to the UVI tube is avoided, and the space requirement of the UVI tubing is minimized, e.g. when incorporated into various devices and technical applications. The connector structure may be incorporated to a construction of a device, an instrument or a technical system or the like, now referring to FIG. 29 showing a simplified schematic illustration of an example of a technical system comprising multiple separate devices, wherein the connector structure 4 is incorporated into two of the devices correspondingly to the principle of the preferred embodiment (referring to FIG. 11). In the case of the example, UV light is produced in a single light source 1 and UV light is delivered to the entity comprising a device and an incorporated connector structure 4 through a system of UV distribution light cables 6.

According to another preferred embodiment, now referring to FIGS. 12-13, UV light is produced in a light source, preferably in a UV light emitting diode (UV-LED) light source that locates in direct contact to the connector structure (FIG. 12) or within the connector structure (FIG. 13). Correspondingly, the connector structure comprises in addition to means for accepting said liquid 2 to be passed into the tube interior 8 for conveyance and means for accepting UV light 1 to pass through one or both open tube ends, means for providing a support (support structure) for the light source in connection to or within the connector structure, and at least one conduit for providing a passage for said liquid substance 2 between the tube interior 8 and the exterior of the connector structure. In the case of the embodiment, the means for accepting UV light 1 to pass into the interior 8 of the tube 3 that are provided in association of the connector structure 4, may for example comprise providing an optical window incorporated to the connector structure, or providing means for positioning the light source itself at the open UVI tube end in a way that the optical connection is achieved. Some benefits of UV-LED sources are low heat production, small size and low power consumption. The connector structure may be incorporated to a construction of a device, an instrument or a technical system or the like, now referring to FIG. 30, showing a simplified schematic illustration of an example of a technical system comprising multiple separate devices, wherein the connector structure 4 is incorporated into two of the devices correspondingly to the principle of a preferred embodiment illustrated in FIG. 13. In the case of the example, UV light is produced in two UV-LED light sources 1 located within the two entities each comprising a device and an incorporated connector structure 4.

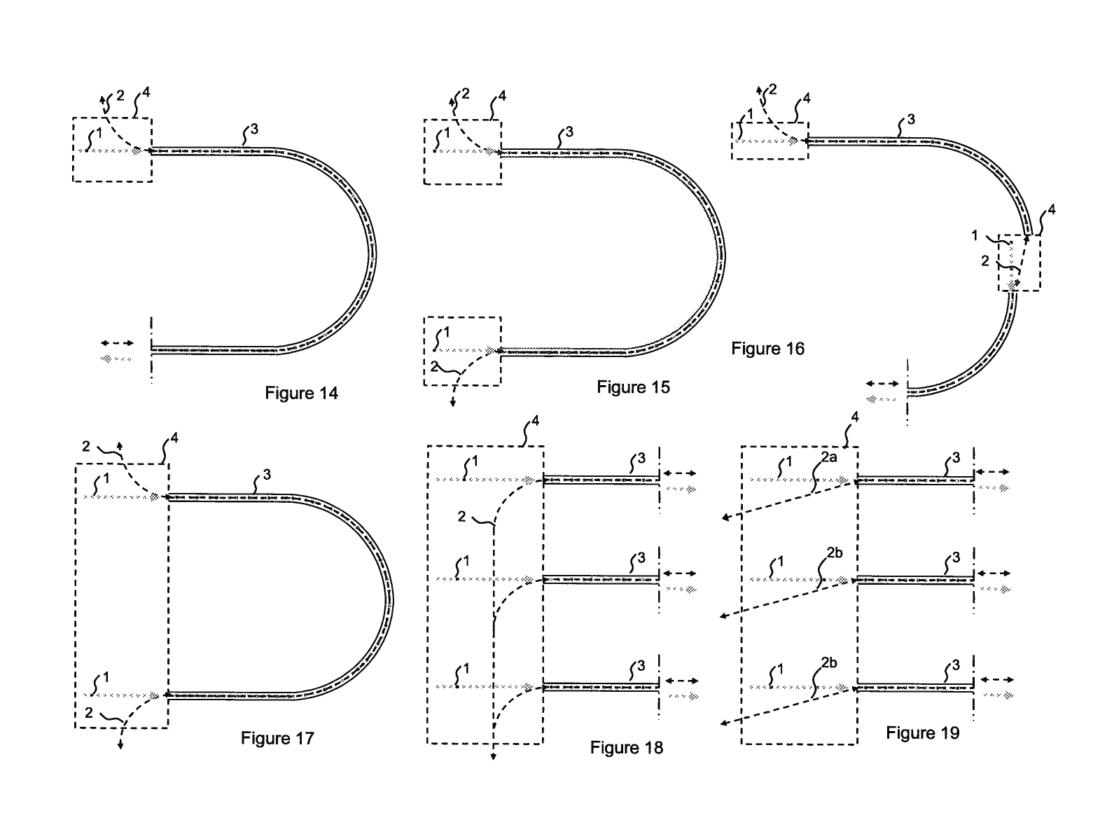

Moreover, now referring to FIGS. 14-19, which illustrate some alternative general configurations of connector structures and their functions (with further reference to FIGS. 11-13 and FIGS. 29-30), the connector structure 4 may further comprise: means for attaching two or a plurality of UVI tubes 3 to the connector structure 4 and means for providing two or a plurality of conduits for conveying at least one liquid substance between the exterior of the connector structure and said interiors of said tubes. Thus, a required amount of passages for one liquid substance 2 (now referring to FIG. 17) or several different liquid substances 2a-2b (now referring to FIG. 18) may be provided in association with the connector structure. To continue, firstly with further reference to the previously described embodiment wherein light source is located at a distance to the connector structure (illustrated in FIG. 11) the connector structure may further comprise means for correspondingly interfacing two or a plurality of UVI tubes 3 and light cables delivering UV light 1 into UVI tubes 3, and secondly with further reference to the previously described embodiment wherein light source is located in contact or within the connector structure (illustrated in FIGS. 12-13), the connector structure may further comprise means for providing a support structure for two or a plurality of light sources (preferably UV-LED light sources) and means for delivering UV light from two or a plurality of light sources into the interiors of UVI tubes. Correspondingly, the connector structure may comprise means for delivering UV light into the interior of the UVI tube from either the plurality of light cables delivering UV light or directly from the plurality of light sources in contact or within the connector structure. At this point, it should be acknowledged, that the illustrations represented in FIGS. 14-19 are meant to illustrate alternative general configurations of both embodiments of the system independent of the location of the light source (now referring to embodiments illustrated in FIG. 11 and FIGS. 12-13).

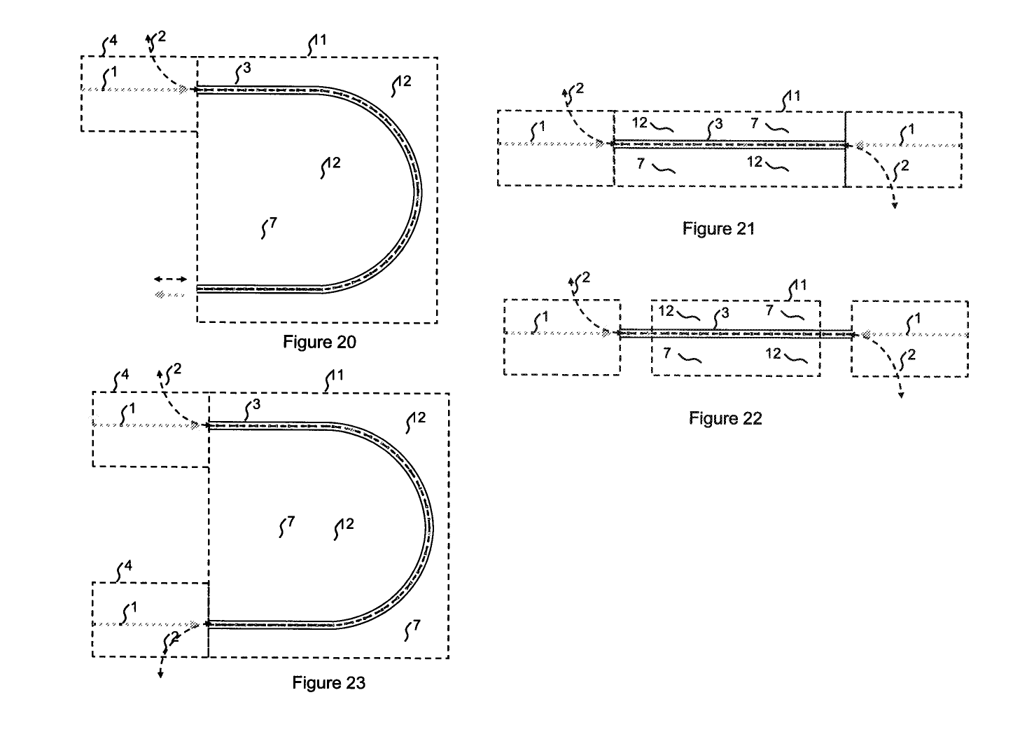

According to another preferred embodiment of the present disclosure, now referring to FIGS. 20-23, the length or a section of the length of UVI tube 3 comprising solely UV transmitting layers (with further reference to tube structures illustrated by examples in FIGS. 7-9) is positioned inside a UV light blocking outer structure 11 forming a hollow volume 12 between the exterior surface of the tube and the outer structure. Moreover, the hollow volume 12 contains a transparent material, which may be preferably be gas 7 or alternatively a liquid material having a lower RI than the conveyed liquid substance. As illustrated in FIGS. 6-8, TIR may take place additionally at the outer surface of the UVI tube 3, resulting in enhanced UV transmittance of the entity formed by the UVI tube and the conveyed liquid. The outer structure 11 may be of its structural details and material composition, of various design, and it may provide adaptation and connection with the UVI tube, the connector structure and larger system entities. The outer structure 11 and the connector structure 4, described previously (referring to FIGS. 11-19), may be a single structure or they may be connected to each others, as schematically illustrated in FIGS. 20-21 and FIG. 23. Another intention associated with the aspects of disclosed embodiments, is that the outer structure 11, similarly as the connector structure 4, may in practice be included within the design of a diversity of various device configurations. In one preferred embodiment, the outer structure is incorporated to a construction of a device, an instrument or a technical system or the like. Referring once more to FIGS. 20-23, it must be understood, that outer structure 11 that is illustrated with a dashed line, illustrates a diversity of various constructions.

The methodological principles and system configurations of the aspects of disclosed embodiments are furthermore combined to yet another functional concept referring especially to a certain embodiments of the present disclosure and to certain UVI tube structure alternatives of the invention. According to an additional embodiment of the present disclosure, concurrent processes of degasification of conveyed liquid substance or dissolving of selected gas into the conveyed liquid substance, are carried out during liquid conveyance and UV treatment within the UVI tube, by designing the UVI tube to comprise one or more concentric layers of gas permeable materials of which at least one layer having a lower RI than the conveyed liquid, and by altering the gas corn-position and pressure of the gas volume surrounding the tube, resulting in gas transfer through the tube wall. Degasification is achieved through creating vacuum atmosphere and dissolving of selected gas through creating an overpressure of gas selected to be dissolved within the gas volume surrounding the tube. The importance and applicability of degasification lies in the fact that, deep UV treatment may photochemically increase the dissolved gas concentrations of the treated liquid, especially those of CO₂ and O₂, which are derived from decomposition of organic compounds. Furthermore, in several processes e.g. in the ultra-pure water production within fine-electronics/semiconductor and pharmaceutical industries, dissolved gases are regarded as contaminants required to be removed. Gasification, in more detail, dissolving of selected gas into the liquid is similarly combined with various processes. For example, in the field of disinfection technology, advanced oxidation processes include dissolving of e.g. O₃ and H₂O₂ into the liquid treated with UV light.

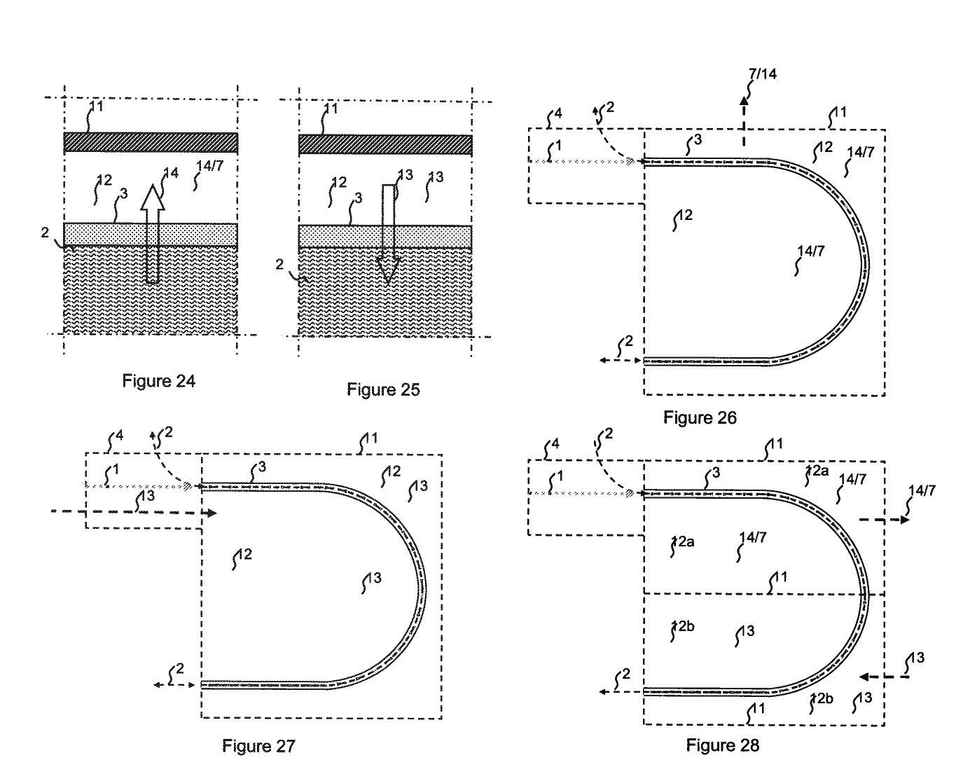

The system corresponding the method with concurrent processes of degasification of or dissolving of selected gas 12 into the liquid substance, now referring to schematic illustrations in FIGS. 24-28, comprises a UVI tube 3, that similarly with the general design of UVI tube structures according to the aspects of disclosed embodiments invention, comprises one or multiple concentric material layers including at least one layer having a lower RI value than to the RI value of the conveyed liquid substance 2, but now in this additional embodiment of the present disclosure, the material or materials of the concentric layer or layers forming the UVI tube are gas permeable polymeric materials. Moreover, now referring to FIGS. 26-28, The UVI tube 3 is enclosed within a solid outer structure 11 (in general described previously in association with FIGS. 20-23), leaving a sealed hollow volume 12 containing air, other gas or gas mixture between the outer surface of UVI tube 3 and the inner surface of the solid outer structure 11. Now referring to FIGS. 24 and 26, degasification of conveyed liquid substance 2 is carried out through providing a vacuum within the sealed hollow volume 12 which surrounds the UVI tube 3 and contains gas material 7 resulting in transfer of dissolved gases 14 contained by the liquid substance 2 to the hollow volume 12 through the gas permeable wall of the UVI tube 3. Thus, a simultaneous UV treatment and degasification of a liquid substance 2 during it's conveyance within the UVI tube 3 is carried out. Now referring to FIGS. 25 and 27, dissolving of gas into the conveyed liquid substance 2 is carried out through providing overpressure of selected gas or a mixture of gases 13 within the sealed hollow volume 12 resulting in transfer of selected gas 13 from the hollow volume 12 through the gas permeable wall of the UVI tube 3 into the UVI tube interior carrying said conveyed liquid substance. Dissolving of selected gas 12 is improved through pre-degasification of the liquid substance. In this perspective, in one embodiment, now referring to FIG. 28, the outer structure 11 and external surface of the UVI tube 3 form two separated sealed spaces 12a and 12b, of which the upstream space 12a is employed for degasification and the downstream space 12b for dissolving of selected gas. As described previously, the solid outer structure 11 may be constructed as connected to the connector structure 4 or the two structures may be constructed as a single structure i.e. within the same construction as the connector structure 4. In the additional embodiment, the solid structure 11 and/or the connector structure 4 comprise at least one gas channel for creation of vacuum conditions or overpressure of selected gas. Thus, the connector structure 4 described previously may in addition to other characterizing features comprise means for enabling creation of said conditions into the sealed hollow volume. In practice, the means may be accomplished through e.g. gas connections (gas channels) between the exterior of the outer structure and the sealed hollow volume through which vacuum and overpressure conditions may be created through various pumping solutions. Required gas channels may be arranged through the outer structure and connector structure. In a preferred embodiment, the UVI tube applied for these additional processes combined to the method and system comprises a single layer of gas permeable polymeric material having a lower RI value than the liquid conveyed within the tube. In a preferred embodiment UVI tube comprises a single layer of Teflon® AF of which gas permeability properties are described previously.

In accordance with embodiments of the present disclosure and as described herein, various modifications and substitutions may be made thereto without losing the central idea, scope and the spirit of the aspects of disclosed embodiments. The description of the aspects of disclosed embodiments has concentrated on illustration and thus does not limit the scope of the disclosed embodiments.

What is claimed is:

1. A method for photochemical treatment of a liquid substances with ultraviolet light the method comprising:
using polymeric tube for conveying said liquid substance, said tube having a first end and a second ends, said tube comprising one or more concentric layers of polymeric materials such that said tube comprises at least one ultraviolet light transmitting polymeric material layer having a refractive index value, which is lower than the refractive index value of said liquid substance,
passing said liquid substance into the interior of said tube through said first end by using said interior of said tube as a conduit for conveying said liquid substance between said first end and said second end, and
directing said ultraviolet light through one or both ends of said tube into the interior of said tube so as to guide said ultraviolet light along said liquid substance by total internal reflection enabled by a difference between the refractive index of the liquid substance and the refractive index of said ultraviolet light transmitting material layer or layers, and photochemically treating said liquid substance in the tube by the ultraviolet light.

2. The method according to claim 1, wherein the refractive index of a ultraviolet light-transmitting material layer of the tube is lower than 1.333 at the wavelength of 589 nm.

3. The method according to claim 1, wherein said tube consists of ultraviolet light transmitting material or materials and the outer surface of said tube is entirely or partially surrounded by gas or liquid material having a refractive index, which is lower than the refractive index of said liquid substance so as to enable total internal reflection of said ultraviolet light by the difference in the refractive index of said material surrounding said tube and said outer surface.

4. The method according to claim 3, wherein said outer surface of said tube is entirely or partially surrounded by gas, said tube comprises a gas permeable material layer or gas permeable material layers, and the method further comprises degasifying said liquid substance through the wall of the tube.

5. The method according to claim 1, wherein said tube further comprises at least one outermost layer of ultraviolet light blocking material.

6. The method according to claim 1, wherein said tube further comprises at least one material layer capable of reflecting ultraviolet light towards the interior of said tube by specular reflection.

7. The method according to claim 1, wherein said ultraviolet light comprises a wavelength or wavelengths in a range from 100 nm to 400 nm.

8. (canceled)

9. The method according to claim 1, wherein said method comprises changing at least one molecular property of said liquid substance by irradiation with the ultraviolet light in the tube.

10. A system for photochemical treatment of a liquid substances with ultraviolet light, the system comprising:
an polymeric tube having a first end and a second, said tube comprising one or more concentric layers of polymer materials such that said tube comprises at least one ultraviolet light transmitting material layer having a refractive index, which is lower than the refractive index of a liquid substance conveyed within said tube,
said system further comprising a ultraviolet light source, which is optically connected to the interior of said tube so as to photochemically treat the liquid substance in the tube by passing ultraviolet light through one or both ends of the tube such that the ultraviolet light is guided along said liquid substance by total internal reflection enabled by a difference between the refractive index of the liquid substance and the refractive index of said ultraviolet light transmitting material layer or layers.

11. The system according to claim 10 further comprising a connector structure connected to an end of said tube.

12. The system according to claim 10 further comprising a light cable arranged to deliver ultraviolet light from the light source into the interior of said tube.

13. The system according to claim 11 further comprising a light source supported by the connector structure.

14-16. (canceled)

17. The system according to claim 10, wherein said tube comprises a single concentric layer of ultraviolet light transmitting polymeric material.

18-20. (canceled)

21. The system according to claim 10, wherein said tube further comprises an outermost layer of ultraviolet light blocking material.

22. The system according to claim 10, wherein the refractive index of at least one of the material layers is lower than 1.333 at the wavelength of 589 nm and at the temperature of 25° C.

23. The system according to claim 10, wherein said tube further comprises at least one material layer capable to reflecting ultraviolet light towards the interior of said tube by specular reflection.

24. The system according to claim 10, wherein said tube or a section of said tube is positioned inside an outer structure forming a hollow volume between the external surface of said tube and said outer structure, said hollow volume containing a transparent gas or liquid material whose refractive index is lower than the refractive index of said liquid substance.

25. The system according to claim 24, further comprising a temperature control unit arranged to control the temperature of the gas or the liquid material within said hollow volume in order to control the temperature of said tube and said liquid substance in the tube.

26. The system according to claim 24, wherein said polymeric material or materials are gas permeable for removing gas from said substance or dissolving gas into said substance.

27-29. (canceled)

30. The method according to claim 3, wherein said outer surface of said tube is entirely or partially surrounded by gas, said tube comprises a gas permeable material layer or gas permeable material layers, and the method further comprises dissolving gas into said liquid substance through the wall of the tube.
